(19) United States
(12) Patent Application Publication
(10) Pub. No.: US 2026/0072025 A1
(43) Pub. Date: Mar. 12, 2026

(54) DETECTION OF HEMOLYSIS USING A CHROMATOGRAPHIC ASSAY DEVICE

(71) Applicant: Siemens Healthcare Diagnostics Inc., Tarrytown, NY (US)

(72) Inventors: Janine Cox, Stoughton, MA (US); David Ledden, Elkhart, IN (US); Jeffrey Jasperse, Newton, MA (US)

(73) Assignee: Siemens Healthcare Diagnostics Inc., Tarrytown, NY (US)

(21) Appl. No.: 18/873,179

(22) PCT Filed: Jun. 14, 2023
(86) PCT No.: PCT/US2023/068454

§ 371 Date: Dec. 9, 2024

Related U.S. Application Data
(60) Provisional application No. 63/366,558, filed on Jun. 17, 2022.

(51) Int. Cl. G01N 33/543; G01N 33/72

(52) U.S. Cl. CPC G01N 33/54388; G01N 33/726; G01N 2333/805; G01N 2800/22

(57) ABSTRACT

Chromatographic assay devices for detecting the presence of free hemoglobin in a liquid biological sample are disclosed. The device comprises a sample application pad and a chromatographic detection pad, wherein the sample application pad is formed of two layers of material. Medical diagnostics devices for use with the chromatographic assay devices are also disclosed, as well as kits and methods of using the chromatographic assay device and/or medical diagnostics device.

### CROSS REFERENCE TO RELATED APPLICATIONS/INCORPORATION BY REFERENCE STATEMENT

This application claims benefit under 35 USC § 119(e) of U.S. Provisional Application No. 63/366,558, filed Jun. 17, 2022. The entire contents of the above-referenced patent application are hereby expressly incorporated herein by reference.

### STATEMENT REGARDING FEDERALLY SPONSORED RESEARCH OR DEVELOPMENT

Not applicable.

### BACKGROUND

Hemolysis refers to the destruction or dissolution of red blood cells (RBCs) which results in the release of hemoglobin (“free hemoglobin”) into surrounding liquid. In the case of a whole blood sample, the free hemoglobin is released into the surrounding plasma. In the case of urine, the free hemoglobin is released into the surrounding water. The occurrence of hemolyzed RBCs may be the result of a patient's medical condition or by the mishandling the sample itself.

Hemolysis is a preanalytical error of concern when testing patient's samples. When severe enough, hemolysis may result in inaccurate laboratory test results. For example, in blood gas and electrolyte testing, it is known that hemolysis will cause an increase in the sample's potassium level. In addition, it is known that cardiac troponin T (cTnT) levels are decreased in samples with hemolysis, and cardiac troponin I (cTnI) levels have been shown to be increased in samples with hemolysis.

The detection of hemolysis in whole blood samples has traditionally been difficult and time consuming. In a central laboratory setting, a whole blood sample is subjected to centrifugation—which generates plasma that is interrogated optically either in the near-infrared (NIR) or visible wavelength regions. While this technique is very effective, it is both complex and time consuming—thereby making this technique ineffective for Point of Care (POC) applications.

In the point of care arena, some systems detect hemolysis electrochemically. However, electrochemical detection of hemoglobin and hematocrit is known to be inaccurate; in addition, hemolysis would only be detected after the electrochemical reagents are consumed, which could waste sample, reagent, and time.

It is important to run whole blood samples as quickly as possible after draw; any delays can impact the sample result. In addition, the samples must be aliquoted and centrifuged before a hemolysis measurement can be made. The requirement for centrifugation, however, limits the settings in which a hemolysis measurement can be determined, and this requirement necessarily lengthens the time between sample collection and testing.

Therefore, there is a need in the art for new and improved devices and methods for hemolysis detection that overcome the disadvantages and defects of the prior art.

### BRIEF DESCRIPTIONS OF THE DRAWINGS

FIG. 1 is a top plan view of one non-limiting embodiment of a chromatographic assay device constructed in accordance with the present disclosure.

FIG. 2 is a cross-sectional view of the chromatographic assay device of FIG. 1.

FIG. 3A is a perspective view of another non-limiting embodiment of a chromatographic assay device constructed in accordance with the present disclosure.

FIG. 3B contains perspective views illustrating a workflow for use of the chromatographic assay device of FIG. 3A.

FIG. 4 schematically depicts one non-limiting embodiment of a reference device utilized with a chromatographic assay device constructed in accordance with the present disclosure.

FIG. 5 schematically depicts another non-limiting embodiment of a reference device utilized with a chromatographic assay device constructed in accordance with the present disclosure.

FIG. 6 schematically depicts one non-limiting embodiment of a medical diagnostics device constructed in accordance with the present disclosure.

### DETAILED DESCRIPTION

Before explaining at least one embodiment of the present disclosure in detail, it is to be understood that the present disclosure is not limited in its application to the details of construction and the arrangement of the components or steps or methodologies set forth in the following description or illustrated in the drawings. The present disclosure is capable of other embodiments or of being practiced or carried out in various ways. Also, it is to be understood that the phraseology and terminology employed herein is for the purpose of description and should not be regarded as limiting in any way.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, compositions, kits, and/or methods disclosed herein can be made and executed without undue experimentation in light of the present disclosure. While the articles, compositions, kits, and/or methods have been described in terms of particular embodiments, it will be apparent to those of skill in the art that variations may be applied to the articles, compositions, kits, and/or methods and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the terms “about” and “approximately” are used to indicate that a value includes the inherent variation of error for a composition/apparatus/device, the method being employed to determine the value, or the variation that exists among the study subjects. That is, the terms “about” and “approximately” and variations thereof are intended to include not only the exact value qualified by the term, but to also include some slight deviations therefrom, such as deviations caused by measuring error, manufacturing tolerances, wear and tear on components or structures, settling or precipitation of cells or particles out of suspension or solution, chemical or biological degradation of solutions over time, stress exerted on structures, and combinations thereof, for example. In particular, for example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps. For example, a composition, a process, method, article, or apparatus that comprises a list of elements is not necessarily limited to only those elements but may include other elements not expressly listed or inherently present therein.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “sample” as used herein will be understood to include any type of biological sample that may be utilized in accordance with the present disclosure. Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

As used herein, the term “liquid sample” and variations thereof is intended to include, for example, but not limited to, biological fluids (such as urine and whole blood), chemical fluids, chemical substances, suspensions, solutions, slurries, mixtures, agglomerations, tinctures, slides, or other preparations of biological fluids, synthetic analogs to biological fluids, and combinations thereof.

Turning now to the various non-limiting embodiments of the inventive concepts, simple, disposable chromatographic assay devices are generally disclosed herein for the fast and efficient detection of hemolysis in liquid samples to inform a medical professional when a sample is compromised and may yield inaccurate test results. The chromatographic assay devices are able to rapidly detect hemolysis in a liquid sample using a small sample size and at a low cost per test. The chromatographic assay devices provide hemolysis detection quickly and free from the requirement of centrifugation, which extends the period from collection to testing and also limits the environment in which the detection test can be performed. Unlike the prior art methods that require centrifugation, the chromatographic assay devices of the present disclosure can be utilized for testing in all types of settings (including central lab and point of care locations, as well as in-home settings). For example (but not by way of limitation), the chromatographic assay devices of the present disclosure can provide results in less than one minute in an inexpensive and versatile manner, and the chromatographic assay devices can be utilized with or without an analyzer in the detection of hemolysis. Visual or optical detection methods can be utilized, which provide more reliable detection.

Certain non-limiting embodiments of the present disclosure are directed to a chromatographic assay device for detecting the presence of free hemoglobin in a liquid biological sample (such as, but not limited to, a whole blood sample, urine, or other red blood cell-containing liquid sample). The device comprises a sample application pad in fluidic contact with a chromatographic detection pad. The sample application pad is configured for application of a portion of the liquid biological sample to the chromatographic assay device. The sample application pad includes at least two different layers of materials. For example (but not by way of limitation), the sample application pad may include a first material layered on or otherwise attached to a second, different material. The at least two different layers of material of the sample application pad may differ in terms of, for example, pore size, uniformity or non-uniformity of pore size, type of material, and/or thickness of material, among other things. In one exemplary embodiment, the sample application pad is formed of a first, upper layer of a prefiltration material and a second, lower layer of a filtration material, where the prefiltration material and the filtration material differ in terms of type of material and/or porosity (e.g., pore size and/or uniformity/non-uniformity of pore size). In certain particular (but non-limiting) embodiments, the sample application pad is formed of a first, upper layer of a prefiltration material and a second, lower layer of an asymmetric filtration material. The term asymmetric filtration material as used herein refers to filtration materials or membranes having asymmetric pore structures where the voids (pores) are non-uniform or vary in size throughout the thickness of the material, such as for example the voids may be smaller near one surface (e.g., the bottom surface) and larger near the opposite surface (e.g., the top surface adjacent to the bottom surface of the prefiltration layer). Exemplary asymmetric filtration materials for use as the second, lower layer may include, for example, polysulfone, polyethersulfone, or mixed cellulose esters. Other exemplary types of material for use as filtration material of the second, lower layer include, for example, potato lectin, or a treated glass fiber membrane treated with, for example, potato lectin. In another illustrative embodiment, the second, lower layer of the sample application pad is formed of a non-asymmetric material, such as a material including potato lectin (e.g., glass fiber treated with potato lectin), while the first, upper layer of a prefiltration material is formed of a different material, such as a material not including potato lectin (e.g., glass fiber not treated with potato lectin).

The sample application pad is porous to plasma and free hemoglobin but not porous to red blood cells, so that red blood cells present in the liquid biological sample are retained within the two layers of material of the sample application pad and are thereby prevented from flowing there through. In particular, but not by way of limitation, the first and/or second layers of material have varying pore sizes that are reduced in size when moving from an upper surface of the sample application pad to the lower surface thereof (i.e., moving in the direction toward the chromatographic detection pad), wherein the first and/or second layers of material have at least one pore size therein that is smaller than the size of a red blood cell, thereby preventing red blood cells from flowing through the sample application pad. In one exemplary embodiment, the first, pre-filtration layer has a less restrictive (e.g., larger) pore size compared to pore size(s) of the second, filtration layer. In another exemplary embodiment, the first, prefiltration layer of material has a more uniform or symmetric pore size structure throughout the thickness of the material compared to the second, filtration layer having a more non-uniform or asymmetric pore size structure.

In addition, the various pore sizes present in the two layers of the sample application pad allow the larger cellular components (i.e., red blood cells) to be retained, but not lysed, while plasma and other free fluids are able to pass through the pores. The combination of the two materials allows the first layer to retain more than the second layer, so that the combination of the two layers allows for retention of all of the red blood cells without overburdening the second layer.

The present disclosure has unexpectedly and advantageously found that a chromatographic assay device comprising a sample application pad with a multi-layered design (e.g., comprised of at least two different materials including a prefiltration material and a filtration material such as an asymmetric filtration material) improves the removal or filtration of red blood cells (RBC) present in the liquid biological sample such that the RBC are trapped within the plurality of layers of the sample application pad, while plasma and free hemoglobin present in the liquid biological sample are allowed to flow (e.g., by gravity) through the pores of the sample application pad to the chromatographic detection pad. Thus, for example, because of the improved filtration of red blood cells by the plurality layers of the sample application pad, the sample application pad is not required to be treated with red blood cell binding or agglutination material. Further, because the multi-layered or dual-layered sample application pad is directly coupled to the chromatographic detection pad, the chromatographic assay device advantageously provides improved removal of RBCs and detection of hemolysis (via detecting the presence of free hemoglobin) in a single or integrated device. Thus, the chromatographic assay device advantageously avoids the need to transfer the plasma and free hemoglobin filtered through the sample application pad to a separate device for detecting the presence or amount of free hemoglobin.

The first layer of the sample application pad may be formed of any material(s) known in the art or otherwise contemplated herein that is capable of retaining at least a portion of the red blood cells and other larger cellular components (without lysing the cells) in a blood sample and thereby reducing the amount of red blood cells and other larger cellular components that flow into the second layer of the sample application pad so as to not overburden the second, filtration layer. One non-limiting example of a material that may be utilized to form the prefiltration layer is a glass fiber material or equivalent; however, it is to be understood that other materials capable of functioning in this manner are known in the art and thus fall within the scope of the term “prefiltration material”as utilized herein.

The chromatographic detection pad defines a path for capillary fluid flow. The chromatographic detection pad has a first end portion and a second end portion. The first end portion is in fluidic contact with the sample application pad and forms a sample application site on the chromatographic detection pad. The chromatographic detection pad also has a detection site that is spaced apart from (and, in certain non-limiting embodiments, downstream of) the first end portion/sample application site, with the detection site being disposed between the first and second end portions or substantially adjacent to the second end portion.

When a liquid biological sample (such as, but not limited to, a whole blood sample, urine, or other red blood cell-containing liquid sample) is applied to the chromatographic assay device, free hemoglobin flows through the sample application pad into the chromatographic detection pad, and then from the sample application site of the chromatographic detection pad to the detection site thereof. In this manner, free hemoglobin (indicative of hemolysis) is detectable at the detection site via a color change.

The chromatographic detection pad may be formed of any material that is white in color (thus allowing for a visual read) and has a pore size that allows for free hemoglobin and plasma to freely flow therethrough whereby the chromatographic detection pad is capable of defining a path of capillary flow for the detection of free hemoglobin in a sample. In a particular (but non-limiting) embodiment, the chromatographic detection pad is formed of a nitrocellulose membrane.

Each of the sample application pad and the chromatographic detection pad may be provided with any pore size that will allow the sample application pad and the chromatographic assay device to function in accordance with the present disclosure. Non-limiting examples of pore sizes that may be utilized in accordance with the present disclosure include about 0.1 micron, about 0.22 micron, about 0.3 micron, about 0.45 micron, about 0.5 micron, about 1 micron, about 2 microns, about 3 microns, about 4 microns, about 5 microns, about 6 microns, about 7 microns, about 8 microns, about 9 microns, about 10 microns, about 11 microns, about 12 microns, about 13 microns, about 14 microns, about 15 microns, about 16 microns, about 17 microns, about 18 microns, about 19 microns, about 20 microns, about 21 microns, about 22 microns, about 23 microns, about 24 microns, about 25 microns, about 26 microns, about 27 microns, about 28 microns, about 29 microns, about 30 microns, about 31 microns, about 32 microns, about 33 microns, about 34 microns, about 35 microns, about 36 microns, about 37 microns, about 38 microns, about 39 microns, about 40 microns, about 41 microns, about 42 microns, about 43 microns, about 44 microns, about 45 microns, about 46 microns, about 47 microns, about 48 microns, about 49 microns, and about 50 microns, as well as a range formed of any of the above values (i.e., a range of from about 0.1 micron to about 6 microns, a range of from 8 microns to about 40 microns, a range of from about 8 microns to about 13 microns, etc.).

In certain particular (but non-limiting) embodiments, the chromatographic assay device may further include a backing material to which the chromatographic detection pad is attached or otherwise associated.

In certain particular (but non-limiting) embodiments, the chromatographic detection pad is a lateral flow strip. In one particular (but non-limiting) embodiment, the lateral flow strip is configured for disposal in or attachment to a medical diagnostics device that detects the color change at the detection site. In another particular (but non-limiting) embodiment, the lateral flow strip is designed for manual use by an individual. In this manner, the color change at the detection site is detected by the individual via a visual comparison to a reference device containing a plurality of reference colors which correspond to different levels of hemolysis.

In certain non-limiting embodiments, the sample application pad may be provided with one or more additional agents that function to further ensure that no red blood cells pass through the sample application pad and into the chromatographic detection pad. For example, but not by way of limitation, the sample application pad may be treated with at least one type of red blood cell (RBC) binding or agglutination material so that the RBC binding or agglutination material exists in a free, soluble form therein. The RBC binding or agglutination material agglutinates with any RBCs in the whole blood sample to produce agglutinated RBCs, wherein the agglutinated RBCs have a size greater than the pore size of the sample application pad and cannot flow through the sample application pad, whereas any free RBC binding or agglutination material can enter the chromatographic detection pad without interfering with the detection of free hemoglobin. Non-limiting examples of RBC binding or agglutination materials that may be utilized in accordance with the present disclosure include a lectin, an anti-human Red Blood Cell (anti-hRBC) binding or agglutination protein antibody, and a human Red Blood Cell (hRBC) binding or agglutination protein that is not an antibody.

The devices and methods of the present disclosure rely on visual or optical detection of hemolysis via a color change in the chromatographic detection pad (such as, but not limited to, a change from the default clear or white color of the pad to a shade of cream, yellow, pink, or red), due to the red color of free hemoglobin. Therefore, in certain non-limiting embodiments, the chromatographic detection pad is devoid of a compound located downstream of the sample application site that is reactive to free hemoglobin in the liquid biological sample.

Alternatively to the chromatographic detection pad being devoid of a reactive compound, the chromatographic detection pad may contain one or more reagents that react with free hemoglobin present in the liquid sample flowing through the chromatographic detection pad. An exemplary reagent present in the chromatographic detection pad may accentuate the color change attributable to free hemoglobin in the detection zone. Non-limiting, exemplary reagents utilize the peroxidase-like activity of hemoglobin, which catalyzes the reaction of diisopropylbenzene dihydroperoxide and 3,3′, 5,5′-tetramethylbenzidine. The resulting color ranges from orange through green and possibly up to blue. Exemplary reagents located in the detection pad may be arranged (for example, but not by way of limitation) into a strip arranged perpendicular to the direction of flow.

Certain non-limiting embodiments of the present disclosure are related to a kit that comprises at least one of any of the chromatographic assay devices described or otherwise contemplated herein above, in combination with a reference device for use by an individual in the visual detection of hemoglobin. The reference device contains a plurality of reference colors, wherein each reference color corresponds to a different level, degree, or amount of hemolysis. The reference device may assume any form or format known in the art or otherwise contemplated herein. For example (but not by way of limitation), the kit may include a container in which the chromatographic assay device(s) is stored prior to use, and a portion of an outer surface of the container may have the reference device attached thereto or formed thereon. Alternatively, the reference device may be an insert card present within the container.

In addition to the chromatographic assay device(s) and reference device, the kits may further contain other component(s) or reagent(s) for use when conducting any of the particular assays described or otherwise contemplated herein. The nature of these additional reagent(s) will depend upon the particular assay format, and identification thereof is well within the skill of one of ordinary skill in the art; therefore, no further description thereof is deemed necessary. Also, the compositions/reagents present in the kits may each be in separate containers/compartments, or various compositions/reagents can be combined in one or more containers/compartments, depending on the reactivity and stability of the compositions/reagents. For example (but not by way of limitation), the kit may further include positive and/or negative control reagents. In addition, the kit may further include a set of written instructions explaining how to use the kit. A kit of this nature can be used in any of the methods described or otherwise contemplated herein.

Certain non-limiting embodiments of the present disclosure are directed to a method of testing a liquid biological sample for hemolysis. In the method, the liquid biological sample is applied to the sample application pad of any of the chromatographic assay devices disclosed or otherwise contemplated herein above, and plasma and any free hemoglobin present in the liquid biological sample is allowed to flow through the sample application pad to the chromatographic detection pad and from the sample application site to the detection site thereof. Then the color change at the detection site is visually compared to a reference device containing a plurality of reference colors, wherein each reference color corresponds to a different level of hemolysis. The visual comparison step may be performed manually by an individual or may be performed by a diagnostics device.

Certain non-limiting embodiments of the present disclosure are directed to a medical diagnostics device assembly. The assembly comprises any of the chromatographic assay devices disclosed or otherwise contemplated herein above in combination with a medical diagnostics device. The medical diagnostics device comprises a light source, an optical sensor, and a processor. The optical sensor detects an amount of red light reflected by the detection site of the chromatographic assay device and outputs a detection signal; the amount of red light reflected by the detection site is attributable to an amount of free hemoglobin present in the liquid biological sample. The processor receives the detection signal and determines the amount of free hemoglobin in the liquid biological sample.

Certain non-limiting embodiments of the present disclosure are directed to a method of testing a liquid biological sample (such as, but not limited to, a whole blood sample, urine, or other red blood cell-containing liquid sample) for hemolysis. In the method, the liquid biological sample is applied to the sample application pad of any of the chromatographic assay devices disclosed or otherwise contemplated herein, and plasma and any free hemoglobin present in the liquid biological sample are allowed to flow through the sample application pad to the chromatographic detection pad and from the sample application site to the detection site thereof. The chromatographic assay device is placed in a medical diagnostics device that comprises a light source, an optical sensor, and a processor, wherein the optical sensor detects an amount of red light reflected by the detection site of the chromatographic assay device and outputs a detection signal, and wherein the processor receives the detection signal and determines the amount of free hemoglobin in the liquid biological sample. An amount of red light reflected by the detection site of the chromatographic assay device is measured, and the amount of red light reflected by the detection site is attributable to an amount of free hemoglobin present in the liquid biological sample. Then the amount of free hemoglobin present in the liquid biological sample is determined based on the measured amount of reflected red light.

In certain particular (but non-limiting) embodiments, the method may further include the step of displaying a notification that the liquid biological sample is hemolyzed when the measured amount of reflected red light exceeds a predefined reference value. When this occurs, in a particular (but non-limiting) embodiment, the method may further include a step of preventing a subsequent test from being performed using the liquid biological sample when the notification that the liquid biological sample is hemolyzed is displayed.

In another particular (but non-limiting) embodiment, the method may further include the steps of: allowing a subsequent test to be performed using the liquid biological sample when the measured amount of reflected red light does not exceed a predefined reference value; and reporting the results of the subsequent test to the healthcare provider.

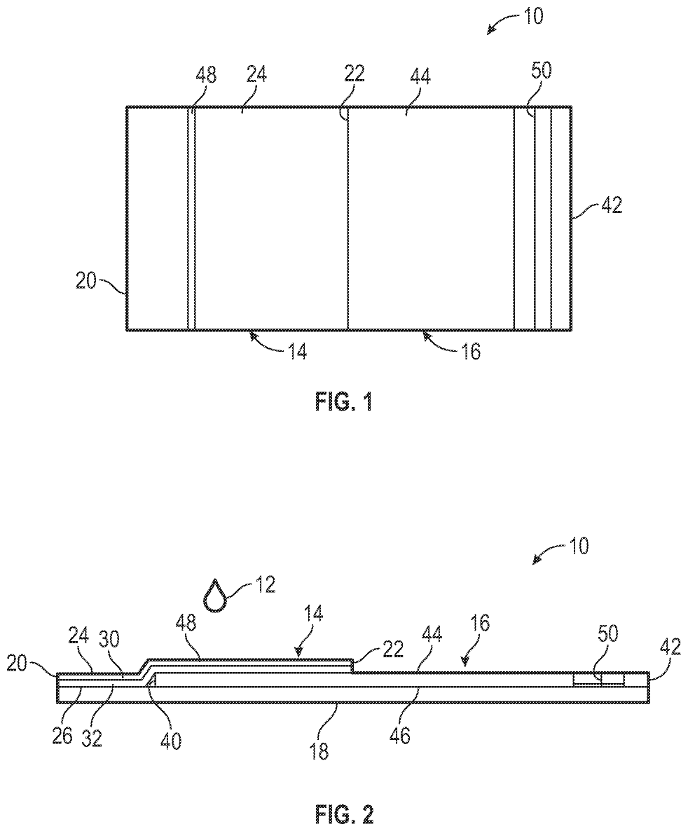

Referring now to FIGS. 1 and 2, a chromatographic assay device 10 for detecting the presence of free hemoglobin in a liquid biological sample 12 (such as, but not limited to, whole blood, urine, or other red blood cell-containing sample) is shown. The chromatographic assay device 10 comprises a sample application pad 14 in fluidic contact with a chromatographic detection pad 16. The sample application pad 14 is configured for application of a portion of the liquid biological sample 12 to the chromatographic assay device 10. The sample application pad 14 may receive and absorb the liquid biological sample 12, and the liquid biological sample 12 may then be absorbed into the chromatographic detection pad 16 from the sample application pad 14.

The sample application pad 14 has a first end 20, a second end 22, an upper surface 24, and a lower surface 26. The sample application pad 14 is formed of two layers of material: a first, upper layer 30 formed of a prefiltration material (such as, but not limited to, a glass fiber material or other suitable prefiltration material) and a second, lower layer 32 formed of an asymmetric filtration material (such as, but not limited to, a polysulfone material or other suitable asymmetric filtration material). However, it should be understood that the second, lower layer 32 may be formed of a non-asymmetric material, where the second, lower layer 32 is formed of a material different than the first, upper layer 30, as discussed above. The first layer 30 of prefiltration material forms the upper surface 24 of the sample application pad 14, while the second layer 32 of asymmetric filtration material forms the lower surface 26 of the sample application pad 14. The first layer 30 has a first surface and a second surface. The first surface of the first layer 30 is the upper surface 24 of the sample application pad 14 configured to receive the liquid biological sample 12, such as (but not limited to) directly receiving the liquid biological sample 12 from, for example (but not by way of limitation), a syringe, pipette, dropper, bulb, capillary, or other dispensing apparatus. The second surface of the first layer 30 is directly formed on and/or directly attached to a first surface of the second layer 32, while the second surface of the second layer 32 is the lower surface 26 of the sample application pad 14 in direct fluidic contact with the chromatographic detection pad 16. Accordingly, in the embodiment shown in FIG. 2, the sample application pad 14 is advantageously formed of a first layer 30 directly formed on and/or directly attached to the second layer 32 without additional intervening layers (excluding means for forming and/or attaching the first layer 30 to the second layer 32).

The sample application pad 14 is porous to plasma and free hemoglobin present in the liquid biological sample 12 but is not porous to red blood cells, so that red blood cells present in the liquid biological sample 12 are retained within the two layers 30 and 32 of material of the sample application pad 14 and are thereby prevented from flowing there through to the chromatographic detection pad 16.

The chromatographic detection pad 16 defines a path for capillary fluid flow. Components of the liquid biological sample 12 that are capable of flowing through the sample application pad 14 then flow through the chromatographic detection pad 16 by capillary action (which may also be referred to as capillary flow). The chromatographic detection pad 16 has a first end portion 40 and a second end portion 42. The chromatographic detection pad 16 may be made of any suitable material that allows plasma and free hemoglobin from the liquid biological sample 12 to freely flow therethrough by capillary action. As one non-limiting example, the chromatographic detection pad 16 may be a nitrocellulose membrane. The chromatographic detection pad 16 may have pores through which certain components of the liquid sample 12 moves by capillary action. The majority of the pores of the chromatographic detection pad 16 may all be substantially the same size or fall within a range of values.

The first end portion 40 of the chromatographic assay device 10 is in fluidic contact with the lower surface 26 of the sample application pad 14 and forms a sample application site 48 on the chromatographic detection pad 16. The chromatographic detection pad 16 also has a detection site 50 that is spaced apart from (and, in certain non-limiting embodiments, downstream of) the first end portion 40/sample application site 48, with the detection site 50 being disposed between the first and second end portions 40 and 42 or substantially adjacent to or closer to the second end portion 42 than the first end portion 40.

In addition, the sample application pad 14 only covers a portion of the chromatographic detection pad 16 adjacent the first end portion 40 and sample application site 48 thereof, but not the detection site 50 thereof; in this manner, the flow of sample through the chromatographic detection pad 16 into the detection site 50 thereof is visible. For example (but not by way of limitation), the second end 22 of the sample application pad 14 is positioned adjacent to or only slightly extending beyond a downstream end of the sample application site 48, so that flow of the sample beyond the sample application site 48 towards the detection site 50 is visible.

The chromatographic assay device 10 may further include a backing material 18 to which the lower surface 46 of the chromatographic detection pad 16 is attached or otherwise associated (such as, but not limited to, via double stick adhesive).

When a liquid biological sample 12 (such as, but not limited to, a whole blood sample, urine, or other red blood cell-containing liquid sample) is applied to the chromatographic assay device 10, free hemoglobin flows through the sample application pad 14 into the chromatographic detection pad 16, and then from the sample application site 48 of the chromatographic detection pad 16 to the detection site 50 thereof. In this manner, free hemoglobin (indicative of hemolysis) is detectable at the detection site via a color change due to the red color of free hemoglobin.

While one particular, non-limiting embodiment of a chromatographic assay device 10 is shown in FIGS. 1-2, it will be understood that the design and configuration of the chromatographic assay device 10 shown is for purposes of example only. The scope of the present disclosure includes adapting the design and configuration of the chromatographic assay devices of the present disclosure, so long as the chromatographic assay device remains capable of functioning in accordance with the present disclosure.

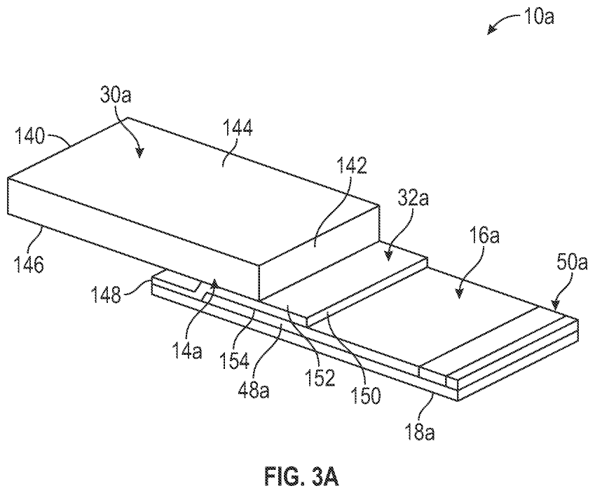

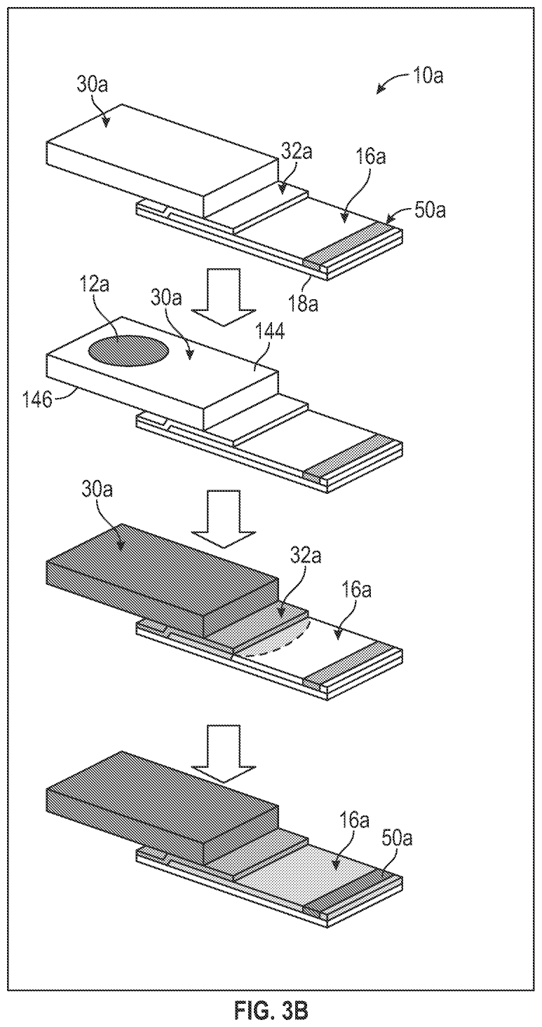

For example (but not by way of limitation), it will be understood that the first layer (of prefiltration material) and the second layer (of asymmetric filtration material) of the sample application pad do not have to be symmetrical with one another (i.e., they can differ from one another in size, length, width, and/or thickness). In addition, the first and second layers of the sample application pad do not have to be congruous with one another, and therefore each layer can have an area that does not overlap with the other layer. The only requirement is that at least a portion of the first layer must overlap a sufficient portion of the second layer so that the sample can flow through the first layer into the second layer and then flow from the second layer into the sample application site of the chromatographic detection pad. FIG. 3A depicts a chromatographic assay device 10a that is similar to the chromatographic assay device 10 of FIGS. 1-2 except that the chromatographic assay device 10a has a sample application pad 14a formed of two separate layers that have different sizes and dimensions and also do not fully overlap with one another. The sample application pad 14a includes a first layer 30a formed of a prefiltration material and a second layer 32a formed of an asymmetric filtration material. The first layer 30a has a first end 140, a second end 142, an upper surface 144, and a lower surface 146. The second layer 32a has a first end 148, a second end 150, an upper surface 152, and a lower surface 154. At least a portion of the first layer 30a adjacent to the second end 142 thereof overlaps a portion of the second layer 32a between the first and second ends 148 and 150 thereof. The overlapping portions of the first and second layers 30a and 32a may be attached to one another, or the overlapping portion of the first layer 30a may simply be layered upon the second layer 32a, so that a portion of the lower surface 146 of the first layer 30a is in contact with a portion of the upper surface 152 of the second layer 32a.

FIG. 3B illustrates a workflow for the chromatographic assay device 10a of FIG. 3A, however it should be noted that a workflow for the chromatographic assay device 10 of FIG. 2 is substantially the same. A blood sample 12a is applied to the first layer 30a of the sample application pad 14a and enters the first layer 30a. While the second panel of FIG. 3B illustrates the blood sample 12a as being applied to the upper surface 144 of the first layer 30a, it will be understood that, when the first and second layers 30a and 32a do not fully overlap with one another, the blood sample 12a may optionally be applied to the lower surface 146 of the first layer 30a in the non-overlapping portion thereof. The blood sample 12a then saturates the first layer 30a and flows through the overlapping portion into the second layer 32a (third panel). Plasma from the blood sample 12a then passes through the second layer 32a and enters the chromatographic detection pad 16a and flows from the sample application site 48a to the detection site 50a thereof for detection of any hemolysis present (fourth panel). In one non-limiting embodiment, the plasma that reaches the detection site 50a turns the control line from yellow to blue, indicating that the device 10a is ready to be read.

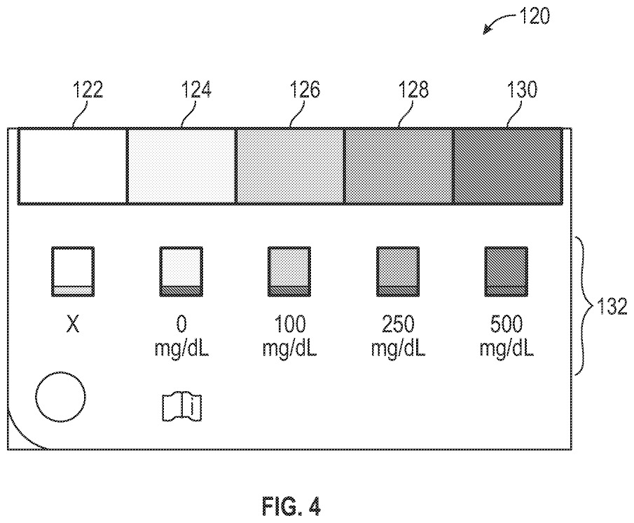

FIG. 4 illustrates one non-limiting embodiment of a reference device 120 that can be utilized in conjunction with a chromatographic assay device 10 to visually determine the level of hemolysis in a liquid sample 12. The reference device 120 contains a plurality of reference colors (such as, but not limited to, the reference colors 122, 124, 126, 128, and 130, with color 122 having the white/default color of the chromatographic detection pad 16 and serving as a negative control, and colors 124, 126, 128, and 130 being various shades of pink/red in increasing intensities/hues, wherein the darker intensities/hues correlate to higher amounts/degrees of hemolysis); five reference colors shown for purposes of illustration only). In addition, the reference device 120 also contains a key 132 that correlates each of the reference colors 122, 124, 126, 128, and 130 to a specific concentration of free hemoglobin. That is (and for purposes of example only), color 122 of the key 132 is the negative control, while color 124 indicates that 0 mg/dl free hemoglobin is present, color 126 indicates that 100 mg/dL free hemoglobin is present, color 128 indicates that 250 mg/dL free hemoglobin is present, and color 130 indicates that 500 mg/dL free hemoglobin is present. In this manner, an individual can determine a level of hemolysis in a liquid biological sample in any setting (including, but not limited to, point-of-care or in-home settings) by comparing the color at the detection site 50 to the reference colors 124-130 of the reference device 120.

The design and configuration of the reference device 120 of FIG. 4 is shown for purposes of example only; it will be understood that the reference device 120 may be provided with less than five reference colors or more than five reference colors thereon (such as, but not limited to, two, three, four, five, six, seven, eight, nine, ten, or more reference colors thereon). In addition, the shapes and placement of the reference colors may be different. Also, the key 132 may be provided with different shapes/placement that differs from that shown in FIG. 4. That is, the design and configuration of each of the various components of the reference device 120 (such as, but not limited to, the reference colors and the key 132) may easily be adapted by a person of ordinary skill in the art to possess any design and configuration that will allow the reference device 120 to function in accordance with the present disclosure.

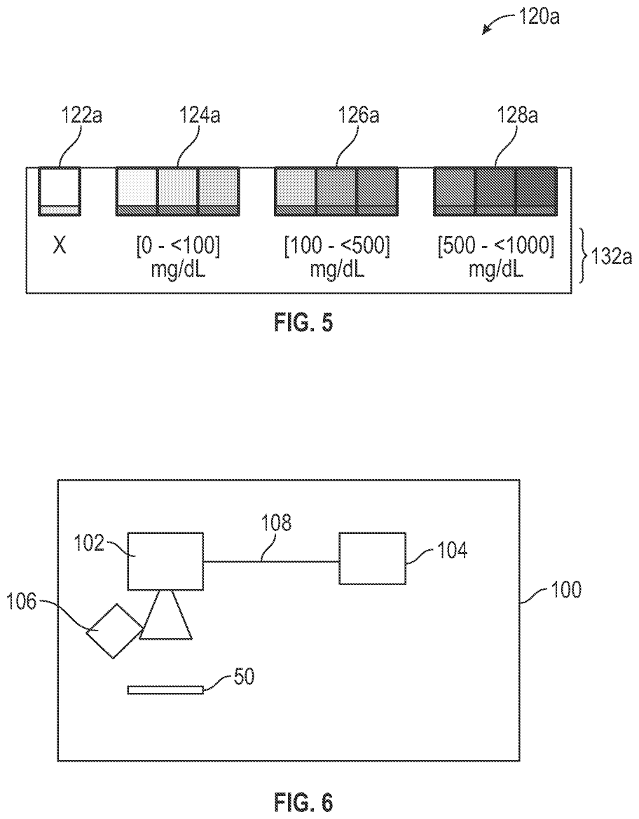

For example (but not by way of limitation), FIG. 5 illustrates another non-limiting embodiment of a reference device (designated herein by reference numeral 120a) that can be utilized in conjunction with a chromatographic assay device 10 to visually determine the level of hemolysis in a liquid sample 12. While the reference device 120 of FIG. 4 contains individual reference colors, the reference device 120a of FIG. 5 contains a plurality of ranges of reference colors (such as, but not limited to, the reference color ranges 122a, 124a, 126a, and 128a, with color 122a having the white/default color of the chromatographic detection pad 16 and serving as a negative control, and color ranges 124a, 126a, and 128a each including a range of various shades of pink/red in increasing intensities/hues, wherein the darker intensities/hues correlate to higher amounts/degrees of hemolysis); four ranges of reference colors shown for purposes of illustration only). The reference device 120a also contains a key 132a that correlates a specific concentration range of free hemoglobin with each of the reference color ranges 122a, 124a, 126a, and 128a. That is (and for purposes of example only), color 122a of the key 132a is the negative control, while color range 124a indicates that 0-<100 mg/dL free hemoglobin is present, color range 126a indicates that 100-<500 mg/dl free hemoglobin is present, and color range 128a indicates that 500-1000 mg/dL free hemoglobin is present. In this manner, an individual can determine a level of hemolysis in a liquid biological sample in any setting (including, but not limited to, point-of-care or in-home settings) by comparing the color at the detection site 50 to the reference color ranges 124a-128a of the reference device 120a.

Alternatively, a medical diagnostics device may be utilized to optically detect the level of hemolysis in a liquid biological sample. Referring now to FIG. 6, a medical diagnostics device 100 is depicted. The medical diagnostics device 100 comprises an optical sensor 102, a processor 104, and a light source 106 directed at the detection site of the chromatographic assay device (such as, but not limited to, the detection site 50 of the chromatographic assay device 10 of FIGS. 1-2). The optical sensor 102 takes one or more images of the detection site 50 and transmits the image(s) to the processor 104 in detection signal(s) 108. The processor 104 then analyzes the characteristics of the light reflected by the detection site 50 of the chromatographic assay device 10 based on the received image(s). The characteristics, such as the observable colors (e.g., red, orange, green, and blue), of the light reflected by the detection site 50 are attributable to the presence of free hemoglobin in the liquid sample 12. Thus, the characteristics of the reflected light can be used by the processor to quantify the amount of free hemoglobin present in the sample liquid 12. For example, when the detection site/zone 50 is devoid of a compound located downstream of the sample application pad 14 or the sample application site 48 of the chromatographic detection pad 16, the amount/intensity of red light reflected by the detection site 50 can be used to quantify the amount of free hemoglobin present in the liquid sample 12. When the chromatographic detection pad 16 contains a reagent(s) that reacts with free hemoglobin and is located downstream of the sample application site 48, the amount/intensity of one or more of red, orange, green, or blue light reflected by the detection site 50 can be used to quantify the amount of free hemoglobin present in the liquid sample 12. Thus, the processor 104 is able to determine the amount of free hemoglobin in the liquid sample 12 by, for example, comparing the measured amounts of the observable colors of the light reflected by the detection site 50 against known reference values. It should also be understood that the processor 104 need not be located within the device 100 and can be located at an external location.

In one non-limiting embodiment, the light source 106 may be a broadband light source, and the optical sensor 102 may employ a two-dimensional array of pixels capturing a two-dimensional image of the detection site 50. The processor 104 may be configured to select specific regions of interest within the image of the chromatographic assay substrate, analyze spectral content and surface topography of the regions of interest on the substrate, determine porosity and depth variation of the regions of interest, algorithmically improve selectivity, dynamic range, and signal to noise of the primary signals of interest, that are otherwise degraded by variations in the detection region, residual sample turbidity and chemical interferents.

A method of testing a liquid sample for hemolysis may include measuring the characteristics of the light reflected by the detection site 50 of the chromatographic assay device 10, as described above, after a portion of the liquid sample 12 has been applied to the sample application pad 14 and free hemoglobin has flowed through the sample application pad 14 and into the chromatographic detection pad 16 from the sample application site 48 to the detection site 50. The measured amount(s) of, for example, red, orange, green, and/or blue light can then be used in determining the level of free hemoglobin by, for example, comparing the measured amount(s) against one or more reference values. In exemplary embodiments, the method may be performed by device 100 or by a medical provider. A medical provider may, for example, compare the completed the chromatographic assay device 10 against a reference device (such as, but not limited to, the reference devices shown in FIGS. 4 and 5), wherein the reference device contains a plurality of reference colors which each correspond to a different level of hemolysis, in order to visually determine the hemolysis of the liquid sample 12.

This method may be used to detect the levels of hemoglobin that exceed a predetermined interference value (for example, a manufacturers'interference level). If the sample is above the interference value, the sample would be flagged to inform the end user (i.e., the relevant healthcare provider) that the sample is hemolyzed and therefore compromised. Where the medical diagnostics device 100 is able to perform additional tests on the liquid sample 12 after determining that the liquid sample 12 is hemolyzed, the device 100 may either prevent a subsequent test from being performed using the liquid sample 12 or allow a subsequent test to be performed using the liquid sample 12 but notify the end user to take into account that the liquid sample 12 is hemolyzed when interpreting the results of the subsequent test(s).

The processor 104 may have any suitable architecture, such as a general processor, central processing unit, digital signal processor, application specific integrated circuit, field programmable gate array, digital circuit, analog circuit, combinations thereof, or any other now known or later developed device for processing data. Likewise, processing strategies may include multiprocessing, multitasking, parallel processing, and the like. A program may be uploaded to, and executed by, the processor. The processor implements the program alone or includes multiple processors in a network or system for parallel or sequential processing.

The processor outputs the state and/or associated information on the display, into a memory, over a network, to a printer, or in another media. The display is text, graphical, or other display.

The display is a CRT, LCD, plasma, projector, monitor, printer, or other output device for showing data. The display is operable to output to a user a state associated with a patient. The state provides an indication of whether a medical concept is indicated in the medical transcript. The state may be whether a disease, condition, symptom, or test result is indicated. In one embodiment, the state is limited to true and false, or true, false and unknown. In other embodiments, the state may be a level of a range of levels or other non-Boolean state.

The processor 104 operates pursuant to instructions. The instructions may be embodied in a program. The program may be a non-transitory computer-readable medium that stores instructions that, when executed by the at least one processor 104 cause the processor 104 to quantify the amount of free hemoglobin present in the liquid sample 12 based on image(s) of the detection site 50 according to any one of the techniques described herein. The program may be located non-transitory a computer readable memory such as an external storage, ROM, and/or RAM. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. In one embodiment, the instructions are stored on a removable media device for reading by local or remote systems. In other embodiments, the instructions are stored in a remote location for transfer through a computer network or over telephone lines, wireless GSM, or WiFi. In yet other embodiments, the instructions are stored within a given computer, CPU, GPU or system. Because some of the constituent system components and method acts depicted in the accompanying figures may be implemented in software, the actual connections between the system components (or the process steps) may differ depending upon the manner of programming.

### Non-Limiting Illustrative Embodiments

The following is a list of non-limiting illustrative embodiments disclosed herein:

Illustrative embodiment 1. A chromatographic assay device for detecting presence of free hemoglobin in a liquid biological sample, the device comprising: a sample application pad for application of the liquid biological sample, wherein the sample application pad is formed of a first layer of a prefiltration material and a second layer of a filtration material which is different than the prefiltration material, wherein the sample application pad is porous to plasma and the free hemoglobin and not porous to red blood cells so that red blood cells present in the liquid biological sample are retained within the first and second layers of the sample application pad and are thereby prevented from flowing there through; and a chromatographic detection pad which defines a path for capillary fluid flow, the chromatographic detection pad having a first end portion that is in fluidic contact with the sample application pad and that forms a sample application site on the chromatographic detection pad, the chromatographic detection pad further having a detection site spaced apart from the sample application site; wherein the free hemoglobin flows through the sample application pad into the chromatographic detection pad and from the sample application site to the detection site and is detectable via a color change at the detection site.

Illustrative embodiment 2. The chromatographic assay device of illustrative embodiment 1, wherein the prefiltration material of the sample application pad comprises a glass fiber material, and wherein the filtration material is an asymmetric material and comprises polysulfone.

Illustrative embodiment 3. The chromatographic assay device of illustrative embodiment 1 or 2, wherein the chromatographic detection pad is formed of a nitrocellulose membrane.

Illustrative embodiment 4. The chromatographic assay device of any of illustrative embodiments 1-3, wherein the chromatographic detection pad has a pore size in a range of from about 8 microns to about 40 microns.

Illustrative embodiment 4A. The chromatographic assay device of any one of the preceding illustrative embodiments, wherein the pore size of the chromatographic detection pad is in a range of from about 8 microns to about 13 microns.

Illustrative embodiment 5. The chromatographic assay device of any one of the preceding illustrative embodiments, wherein the chromatographic detection pad is a lateral flow strip.

Illustrative embodiment 6. The chromatographic assay device of any one of the preceding illustrative embodiments, wherein the lateral flow strip is configured for disposal in a medical diagnostics device that detects the color change at the detection site.

Illustrative embodiment 7. The chromatographic assay device of any one of illustrative embodiments 1-6, wherein the chromatographic detection pad is configured to provide a visual comparison of the color change at the detection site to a reference device containing a plurality of reference colors which correspond to different levels of hemolysis.

Illustrative embodiment 8. The chromatographic assay device of any one of illustrative embodiments 1-7, wherein the sample application pad is treated with at least one type of red blood cell (RBC) binding or agglutination material.

Illustrative embodiment 9. The chromatographic assay device of any one of the preceding illustrative embodiments, wherein the RBC binding or agglutination material comprises a lectin.

Illustrative embodiment 10. The chromatographic assay device any one of the preceding illustrative embodiments, wherein the RBC binding or agglutination material comprises an anti-human Red Blood Cell (anti-hRBC) binding or agglutination protein antibody.

Illustrative embodiment 11. The chromatographic assay device of any one of the preceding illustrative embodiments, wherein the RBC binding or agglutination material is a human Red Blood Cell (hRBC) binding or agglutination protein that is not an antibody.

Illustrative embodiment 12. The chromatographic assay device of any one of illustrative embodiments 1-11, wherein the chromatographic detection pad is devoid of a compound located downstream of the sample application site that is reactive to the free hemoglobin in the liquid biological sample, and wherein the free hemoglobin is detected via a red color change at the detection site.

Illustrative embodiment 13. A kit, comprising: at least one chromatographic assay device of any one of illustrative embodiments 1-12; and a reference device containing a plurality of reference colors, wherein each reference color corresponds to a different level of hemolysis.

Illustrative embodiment 14. A method of testing a liquid biological sample for hemolysis, the method comprising the steps of: applying the liquid biological sample to the sample application pad of the chromatographic assay device of any one of illustrative embodiments 1-13 and allowing the plasma and the free hemoglobin present in the liquid biological sample to flow through the sample application pad to the chromatographic detection pad and from the sample application site to the detection site thereof; and visually comparing the color change at the detection site to a reference device containing a plurality of reference colors, wherein each reference color corresponds to a different level of hemolysis.

Illustrative embodiment 15. A medical diagnostics device assembly, the assembly comprising: the chromatographic assay device of any one of illustrative embodiments 1-14; and a medical diagnostics device, the medical diagnostics device comprising: a light source; an optical sensor that detects an amount of red light reflected by the detection site of the chromatographic assay device and outputs a detection signal, the amount of red light reflected by the detection site being attributable to an amount of the free hemoglobin present in the liquid biological sample; and a processor that receives the detection signal and determines the amount of the free hemoglobin in the liquid biological sample.

Illustrative embodiment 16. A method of testing a liquid biological sample for hemolysis, the method comprising the steps of: applying the liquid biological sample to the sample application pad of the chromatographic assay device of any one of illustrative embodiments 1-15 and allowing the plasma and the free hemoglobin present in the liquid biological sample to flow through the sample application pad to the chromatographic detection pad and from the sample application site to the detection site thereof; placing the chromatographic assay device in a medical diagnostics device that comprises a light source, an optical sensor, and a processor, wherein the optical sensor detects an amount of red light reflected by the detection site of the chromatographic assay device and outputs a detection signal, and wherein the processor receives the detection signal and determines an amount of the free hemoglobin in the liquid biological sample; measuring the amount of red light reflected by the detection site of the chromatographic assay device, wherein the amount of red light reflected by the detection site is attributable to the amount of the free hemoglobin present in the liquid biological sample; and determining the amount of the free hemoglobin present in the liquid biological sample based on the measured amount of reflected red light.

Illustrative embodiment 17. The method of any one of the preceding illustrative embodiments, further comprising the step of: displaying a notification that the liquid biological sample is hemolyzed when the measured amount of reflected red light exceeds a predefined reference value.

Illustrative embodiment 18. The method of any one of the preceding illustrative embodiments, further comprising the step of: preventing a subsequent test from being performed using the liquid biological sample when the notification that the liquid biological sample is hemolyzed is displayed.

Illustrative embodiment 19. The method of any one of the preceding illustrative embodiments, further comprising the steps of: allowing a subsequent test to be performed using the liquid biological sample when the measured amount of reflected red light does not exceed a predefined reference value; and reporting a result of the subsequent test to the healthcare provider.

Illustrative embodiment 20. The method of any one of the preceding illustrative embodiments, wherein the liquid biological sample is one of a whole blood sample and urine.

Thus, in accordance with the present disclosure, there have been provided devices and kits, as well as methods of producing and using same, which fully satisfy the objectives and advantages set forth hereinabove. Although the present disclosure has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A chromatographic assay device for detecting presence of free hemoglobin in a liquid biological sample, the device comprising:
a sample application pad for application of the liquid biological sample, wherein the sample application pad is formed of a first layer of a prefiltration material and a second layer of a filtration material which is different than the prefiltration material, wherein the sample application pad is porous to plasma and the free hemoglobin and not porous to red blood cells so that red blood cells present in the liquid biological sample are retained within the first and second layers of the sample application pad and are thereby prevented from flowing there through; and
a chromatographic detection pad which defines a path for capillary fluid flow, the chromatographic detection pad having a first end portion that is in fluidic contact with the sample application pad and that forms a sample application site on the chromatographic detection pad, the chromatographic detection pad further having a detection site spaced apart from the sample application site;
wherein the free hemoglobin flows through the sample application pad into the chromatographic detection pad and from the sample application site to the detection site and is detectable via a color change at the detection site.

2. The chromatographic assay device of claim 1, wherein the prefiltration material of the sample application pad comprises a glass fiber material, and wherein the filtration material is an asymmetric material and comprises polysulfone.

3. The chromatographic assay device of claim 1, wherein the chromatographic detection pad is formed of a nitrocellulose membrane.

4. The chromatographic assay device of claim 1, wherein the chromatographic detection pad has a pore size in a range of from about 8 microns to about 40 microns.

5. The chromatographic assay device of claim 1, wherein the chromatographic detection pad is a lateral flow strip.

6. The chromatographic assay device of claim 5, wherein the lateral flow strip is configured for disposal in a medical diagnostics device that detects the color change at the detection site.

7. The chromatographic assay device of claim 1, wherein the chromatographic detection pad is configured to provide a visual comparison of the color change at the detection site to a reference device containing a plurality of reference colors which correspond to different levels of hemolysis.

8. The chromatographic assay device of claim 1, wherein the sample application pad is treated with at least one type of red blood cell (RBC) binding or agglutination material.

9. The chromatographic assay device of claim 8, wherein the RBC binding or agglutination material comprises a lectin.

10. The chromatographic assay device of claim 8, wherein the RBC binding or agglutination material comprises an anti-human Red Blood Cell (anti-hRBC) binding or agglutination protein antibody.

11. The chromatographic assay device of claim 8, wherein the RBC binding or agglutination material is a human Red Blood Cell (hRBC) binding or agglutination protein that is not an antibody.

12. The chromatographic assay device of claim 1, wherein the chromatographic detection pad is devoid of a compound located downstream of the sample application site that is reactive to the free hemoglobin in the liquid biological sample, and wherein the free hemoglobin is detected via a red color change at the detection site.

13. A kit, comprising:
at least one chromatographic assay device of claim 1; and
a reference device containing a plurality of reference colors, wherein each reference color corresponds to a different level of hemolysis.

14. A method of testing a liquid biological sample for hemolysis, the method comprising the steps of:
applying the liquid biological sample to the sample application pad of the chromatographic assay device of claim 1 and allowing the plasma and the free hemoglobin present in the liquid biological sample to flow through the sample application pad to the chromatographic detection pad and from the sample application site to the detection site thereof; and
visually comparing the color change at the detection site to a reference device containing a plurality of reference colors, wherein each reference color corresponds to a different level of hemolysis.

15. A medical diagnostics device assembly, the assembly comprising:
the chromatographic assay device of claim 1; and
a medical diagnostics device, the medical diagnostics device comprising:
a light source;
an optical sensor that detects an amount of red light reflected by the detection site of the chromatographic assay device and outputs a detection signal, the amount of red light reflected by the detection site being attributable to an amount of the free hemoglobin present in the liquid biological sample; and
a processor that receives the detection signal and determines the amount of the free hemoglobin in the liquid biological sample.

16. A method of testing a liquid biological sample for hemolysis, the method comprising the steps of:
applying the liquid biological sample to the sample application pad of the chromatographic assay device of claim 1 and allowing the plasma and the free hemoglobin present in the liquid biological sample to flow through the sample application pad to the chromatographic detection pad and from the sample application site to the detection site thereof;
placing the chromatographic assay device in a medical diagnostics device that comprises a light source, an optical sensor, and a processor, wherein the optical sensor detects an amount of red light reflected by the detection site of the chromatographic assay device and outputs a detection signal, and wherein the processor receives the detection signal and determines an amount of the free hemoglobin in the liquid biological sample;
measuring the amount of red light reflected by the detection site of the chromatographic assay device, wherein the amount of red light reflected by the detection site is attributable to the amount of the free hemoglobin present in the liquid biological sample; and
determining the amount of the free hemoglobin present in the liquid biological sample based on the measured amount of reflected red light.

17. The method of claim 16, further comprising the step of:
displaying a notification that the liquid biological sample is hemolyzed when the measured amount of reflected red light exceeds a predefined reference value.

18. The method of claim 17, further comprising the step of:
preventing a subsequent test from being performed using the liquid biological sample when the notification that the liquid biological sample is hemolyzed is displayed.

19. The method of claim 16, further comprising the steps of:
allowing a subsequent test to be performed using the liquid biological sample when the measured amount of reflected red light does not exceed a predefined reference value; and
reporting a result of the subsequent test to the healthcare provider.

20. The method of claim 16, wherein the liquid biological sample is one of a whole blood sample and urine.
